(19) United States
(12) Patent Application Publication
(10) Pub. No.: US 2006/0052747 A1
(43) Pub. Date: Mar. 9, 2006

(54) Two-chamber pre-filled syringe

(75) Inventors: Masato Nishimura, Yamanashi (JP); Kouichi Tachikawa, Yamanashi (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Terumo Kabushiki Kaisha, Tokyo (JP)

(21) Appl. No.: 10/496,961

(22) PCT Filed: Oct. 30, 2002
(86) PCT No.: PCT/JP02/11322

§ 371 Date: Mar. 18, 2005

(30) Foreign Application Priority Data
Nov. 22, 2001 (JP) 2001-361393

(51) Int. Cl. A61M 37/00

(52) U.S. Cl. 604/85; 604/86

(57) ABSTRACT

A two-chamber type pre-filled syringe (1) includes an outer cylinder (2) having a projected portion (14b) for breaking use provided inside the outer cylinder (2); an inner cylinder assemblage (20) having an inner cylinder (5), an annular first gasket (6) provided in the vicinity of a front-end portion of the inner cylinder (5), and a sealing member (13) which airtightly seals a front-end portion of the inner cylinder (5) and can be broken by the projected portion (14b) for breaking use; a second gasket (7) accommodated inside the inner cylinder (5); a plunger (8) mounted at a rear-end portion of the second gasket (7); a first accommodation portion (9); a second accommodation portion (11); a medicine-dissolving liquid (10) accommodated in the first accommodation portion (9); and a powdery or frozen dry medicine (12) accommodated inside the second accommodation portion (11).

### TECHNICAL FIELD

The present invention relates to a two-chamber type pre-filled syringe. More particularly, the present invention relates to a two-chamber type pre-filled syringe which accommodates a powdery or frozen medicine separately from a medicine-dissolving liquid for dissolving the medicine therein and is capable of administering the medicine to a patient after the medicine is dissolved in the medicine-dissolving liquid when the two-chamber type pre-filled syringe is used.

### BACKGROUND ART

In a pre-filled syringe in which a medicine is filled, when a plurality of components constituting an injection medicine is mixed with each other, they are liable to decompose or modify in a short period of time. Thus in a known two-chamber pre-filled syringe, the inside of the pre-filled syringe is divided into two chambers to accommodate predetermined components separately from a dissolving liquid, a dispersing liquid or other liquid components.

The two-chamber pre-filled syringe is demanded to make it difficult for a liquid to leak therefrom during transport and preparation for use and allowing a mixing operation to be accomplished easily and securely when the two-chamber pre-filled syringe is used.

Therefore it is an object of the present invention to provide a two-chamber pre-filled syringe making it difficult for a liquid to leak therefrom during transport and preparation for use and allowing a mixing operation to be performed easily and securely when it is used.

### DISCLOSURE OF THE INVENTION

The present invention provides a two-chamber type pre-filled syringe including an outer cylinder having a needle-mounting portion at a front-end side thereof and a projected portion for breaking use provided inside the outer cylinder at the front-end side thereof and projecting toward a rear end thereof; a closing member mounted on the needle-mounting portion; an inner cylinder assemblage comprising a cylindrical inner cylinder accommodated inside the outer cylinder and being open at both ends thereof, an annular first gasket provided in the vicinity of a front-end portion of the inner cylinder and slidable inside the outer cylinder, and a sealing member which airtightly seals a front-end portion of the inner cylinder or a path of the first gasket and can be broken by the projected portion for breaking use provided inside the outer cylinder; a second gasket sidably accommodated inside the inner cylinder; a plunger which is mounted or can be mounted at a rear-end portion of the second gasket; a first accommodation portion formed among the sealing member, the second gasket, and the inner cylinder; a second accommodation portion formed between the inner cylinder assemblage and the outer cylinder; a medicine-dissolving liquid accommodated in the first accommodation portion; and a powdery or frozen dry medicine accommodated inside the second accommodation portion.

### BRIEF DESCRIPTION OF THE DRAWINGS

FIG. 1 is an outlook view of a two-chamber type pre-filled syringe according to an embodiment of the present invention.

FIG. 2 is a sectional view of the two-chamber type pre-filled syringe shown in FIG. 1.

FIG. 3 is an explanatory view for explaining the operation of the two-chamber type pre-filled syringe shown in FIG. 1.

FIG. 4 is an explanatory view for explaining the operation of the two-chamber type pre-filled syringe shown in FIG. 1.

FIG. 5 is an explanatory view for explaining the operation of the two-chamber type pre-filled syringe shown in FIG. 1.

FIG. 6 is an explanatory view for explaining the operation of the two-chamber type pre-filled syringe shown in FIG. 1.

FIG. 7 is a perspective view showing one embodiment of a projected portion for breaking use which is used in the two-chamber type pre-filled syringe shown in FIG. 1.

FIG. 8 is a perspective view showing another embodiment of a projected portion for breaking use which is used in the two-chamber type pre-filled syringe shown in FIG. 1.

FIG. 9 is a perspective view showing another embodiment of a projected portion for breaking use which is used in the two-chamber type pre-filled syringe shown in FIG. 1.

FIG. 10 is a perspective view showing another embodiment of a projected portion for breaking use which is used in the two-chamber type pre-filled syringe shown in FIG. 1.

FIG. 11 is a perspective view showing another embodiment of a projected portion for breaking use which is used in the two-chamber type pre-filled syringe shown in FIG. 1.

FIG. 12 is a perspective view showing another embodiment of a projected portion for breaking use which is used in the two-chamber type pre-filled syringe shown in FIG. 1.

FIG. 13 is an outlook view of a two-chamber type pre-filled syringe according to another embodiment of the present invention.

FIG. 14 is a sectional view of the two-chamber type pre-filled syringe shown in FIG. 13.

FIG. 15 is an explanatory view for explaining the operation of the two-chamber type pre-filled syringe shown in FIG. 13.

FIG. 16 is an explanatory view for explaining the operation of the two-chamber type pre-filled syringe shown in FIG. 13.

FIG. 17 is an explanatory view for explaining the operation of the two-chamber type pre-filled syringe shown in FIG. 13.

FIG. 18 is an explanatory view for explaining the operation of the two-chamber type pre-filled syringe shown in FIG. 13.

FIG. 19 is a perspective view of a projected portion for breaking use which is used in the two-chamber type pre-filled syringe shown in FIG. 13.

FIG. 20 is an outlook view of a two-chamber type pre-filled syringe according to another embodiment of the present invention.

FIG. 21 is a sectional view of the two-chamber type pre-filled syringe shown in FIG. 20.

FIG. 22 is an explanatory view for explaining the operation of the two-chamber type pre-filled syringe shown in FIG. 20.

FIG. 23 is an explanatory view for explaining the operation of the two-chamber type pre-filled syringe shown in FIG. 20.

FIG. 24 is an explanatory view for explaining the operation of the two-chamber type pre-filled syringe shown in FIG. 20.

FIG. 25 is an explanatory view for explaining the operation of the two-chamber type pre-filled syringe shown in FIG. 20.

### BEST MODE FOR CARRYING OUT THE INVENTION

The two-chamber type pre-filled syringe of the present invention will be described below by using the embodiments shown by the drawings.

A two-chamber type pre-filled syringe 1 of the present invention includes an outer cylinder 2 having a needle-mounting portion 21 and a projected portion 14b for breaking use provided inside the outer cylinder 2 and projecting toward a rear end thereof; a closing member mounted on the needle-mounting portion 21; an inner cylinder assemblage comprising a cylindrical inner cylinder 5 accommodated inside the outer cylinder 2 and being open at both ends thereof, an annular first gasket 6 provided in the vicinity of a front-end portion of the inner cylinder 5 and slidable inside the outer cylinder 2, and a sealing member 13 which airtightly seals a front-end portion of the inner cylinder 5 and can be broken by the projected portion 14b for breaking use provided inside the outer cylinder 2; a second gasket 7 sidably accommodated inside the inner cylinder 5; a plunger 8 which is mounted or can be mounted at a rear-end portion of the second gasket 7; a first accommodation portion 9 formed among the sealing member 13, the second gasket 7, and the inner cylinder 5; a second accommodation portion 11 formed between the inner cylinder assemblage 20 and the outer cylinder 2; a medicine-dissolving liquid 10 accommodated in the first accommodation portion 9; and a powdery or frozen dry medicine 12 accommodated inside the second accommodation portion 11.

The two-chamber type pre-filled syringe 1 of this embodiment has the outer cylinder 2, the inner cylinder 5, the plunger 8, and a sealing cap 4.

The outer cylinder 2 is cylindrical. The needle-mounting portion 21 being open at a front end thereof and tapered toward its front end is disposed at the front end of the outer cylinder 2. A pair of flanges 22 is disposed at the rear end of the outer cylinder 2 with the flanges 22 confronting each other. A male screw portion 23 that engages a female screw portion 24 formed on the inner peripheral surface of the sealing cap 4 is formed on the outer surface of the base portion of the needle-mounting portion 21.

As the constituent material for the outer cylinder 2, transparent or semitransparent materials such as polypropylene, annular polyolefin, polycarbonate, polyester, polymethyl pentene, glass, and the like are preferable. As the constituent material for the outer cylinder 2, a material having a low vapor permeability and a material having a low oxygen permeability are preferable to favorably preserve the powdery or frozen dry medicine 12 accommodated in the second accommodation portion 11 formed between the inner cylinder assemblage 20 and the outer cylinder 2. On the other hand, when a desiccant is not enclosed inside the second accommodation portion 11, together with the medicine 12, the material having a low vapor permeability is preferable as the constituent material for the outer cylinder 2. When the outer cylinder 2 is made of a synthetic resin, it is preferable to evaporate or layer silicon oxide over the surface of the outer cylinder 2 or over an intermediate layer thereof. It is suitable that the length of the outer cylinder 2 is 50 to 200 mm. It is suitable that the volume of the outer cylinder 2 is 1.0 to 60 ml.

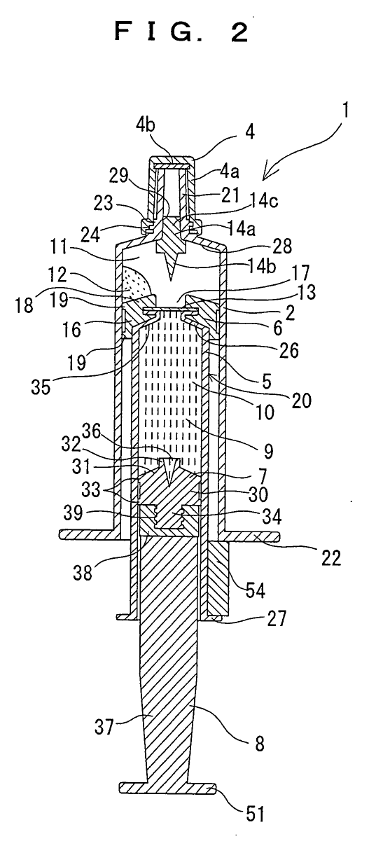

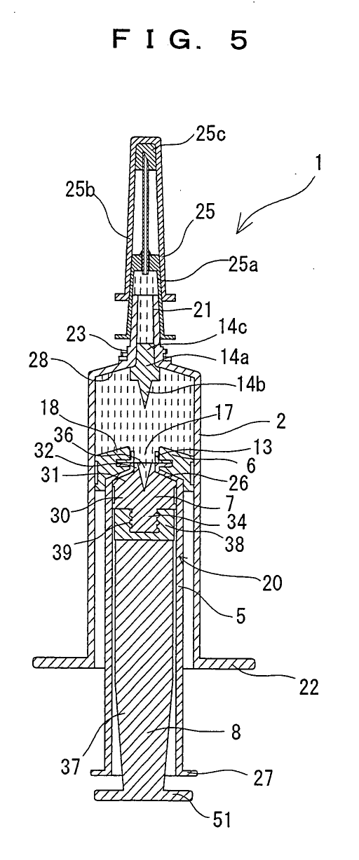

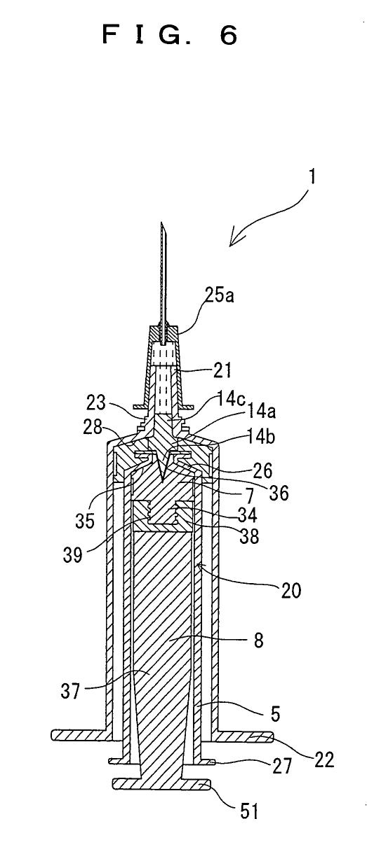

As the closing member to be mounted on the needle-mounting portion 21 of the outer cylinder 2, the sealing cap 4 and a needle 25 in which a front end opening of a needle pipe is sealed with a cap 25b are available. As shown in FIG. 2, the sealing cap 4 has a body portion 4a and a sealing member 4b disposed under a front-end surface of the body portion 4a. In this embodiment, the front end of the body portion 4a is closed. On the inner surface of the base portion of the body portion 4a, the female screw portion 24 which engages the male screw portion 23 formed on the outer surface of the front-end portion of the outer cylinder 2 is formed. Both screws engage each other strongly. Therefore the sealing member 4b contacts the front-end surface of the outer cylinder 2 closely, thus sealing the front end opening (the front end opening of the needle-mounting portion 21) of the outer cylinder 2 airtightly. As shown in FIGS. 5 and 6, as the needle 25, a known needle including a needle body 25a which has a needle pipe having a piercing blade surface at its front-end portion and a hub mounted at the rear-end portion of the needle pipe, and a cap 25b which covers the needle body 25b and whose rear end fits on the hub is used.

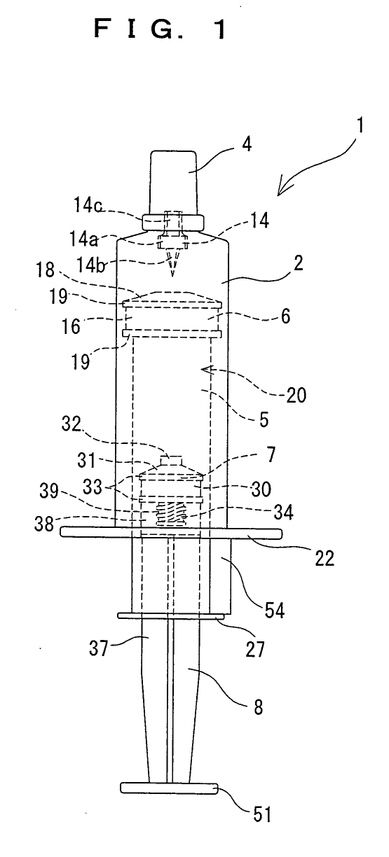

To prevent leak of a liquid medicine accommodated inside the outer cylinder 2 from the front end opening of the needle pipe into the cap 25b, a sealing material 25c for covering the front end opening of the needle pipe is disposed inside the front-end portion of the cap 25b. In the pre-filled syringe shown in FIG. 1, in an unused state, the sealing cap 4 is mounted on the needle-mounting portion 21 and replaced with the needle 25 when the pre-filled syringe is used. But the needle 25 may be initially mounted on the needle-mounting portion 21. The sealing cap 4 may be of a type in which the sealing cap is not removed from the needle-mounting portion 21 but a double ended needle can be directly mounted thereon. For example, the sealing cap may have a body portion having an opening at the center of its front-end portion and a pierceable sealing member so provided as to close the opening of the body portion. By using the double ended needle, it is possible to safely inject a hazardous medicine (chemically hazardous) such as a carcinostatic or mix components of a medicine in a closed state. Further when components of the medicine are mixed with each other by exchanging a plurality of pre-filled syringes at a piercing portion disposed at the rear-end side of the double ended needle, with a piercing portion disposed at the front-end side thereof pierced into a transfusion bag, it is preferable to mount a rubber cover on the double ended needle by covering the piercing portion disposed at the rear-end side thereof with the rubber cover. Thereby when the pre-filled syringes are replaced with each other, it is possible to prevent a transfusion from flowing backward or overflowing from the transfusion bag, prevent the interior thereof from being contaminated because the interior thereof does not communicate with outside air.

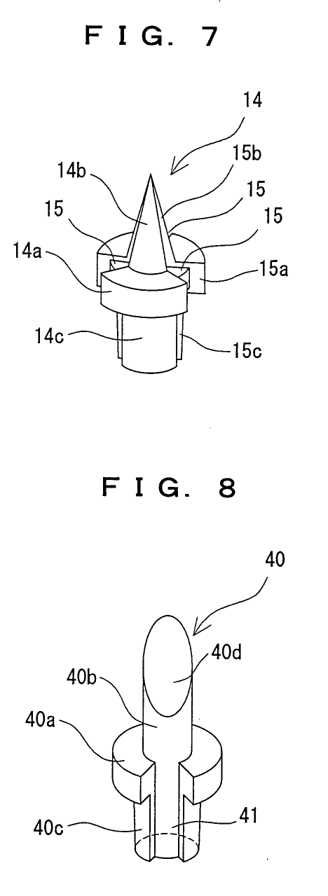

As shown in FIGS. 2 and 7, the two-chamber type pre-filled syringe 1 has a projected member 14 for breaking use mounted on an inner open portion 29 of the needle-mounting portion 21 disposed inside the outer cylinder 2. The projected member 14 for breaking use has a projected portion 14b for breaking use extending toward the rear-end side of the outer cylinder 2, a mounting portion 14c to be mounted on the outer cylinder 2, and a liquid medicine guide path 15 for guiding the liquid medicine from the side of the projected portion 14b for breaking use to the side of the needle-mounting portion 21. The projected portion 14b for breaking use projects from the neighborhood of the inner open portion 29 of the needle-mounting portion 21 of the outer cylinder 2 toward the rear-end side of the outer cylinder 2 and has a sealing member-breaking pointed-end portion disposed at the rear end thereof. The liquid medicine inside the syringe 1 is capable of flowing from the side of the projected portion 14b for breaking use to the side of the needle-mounting portion 21 through the liquid medicine guide path 15.

More specifically, as shown in FIG. 7, the projected member 14 for breaking use is made of a material different from that of the outer cylinder 2 and solid. The projected member 14 for breaking use has a disk-shaped body portion 14a; the needle-shaped projected portion 14b for breaking use (the projected portion for breaking use having the sealing member-breaking pointed-end portion disposed at the rear end thereof) disposed at the rear-end side (upper side in FIG. 7) of the body portion 14a; the rod-shaped mounting portion 14c, disposed at the front-end side (lower side in FIG. 7) of the body portion 14a in such a way that the diameter thereof decreases taperingly toward the front-end side of the outer cylinder 2; and a groove portion (liquid medicine guide path) 15 extending axially from the rear end of the projected portion 14b for breaking use to the front end of the mounting portion 14c along a side surface of the projected member 14 for breaking use. The projected member 14 for breaking use is fixed to the outer cylinder 2 by fitting the mounting portion 14c into the inner open portion 29 of the needle-mounting portion 21.

The groove portion 15 has a groove-forming portion 15b, triangular in its sectional configuration, which extends axially and linearly along a side surface of the projected portion 14b for breaking use; a groove-forming portion 15a formed continuously with the groove-forming portion 15b along the upper, side, and lower surfaces of the disk-shaped body portion 14a; and a groove-forming portion 15c, triangular in its sectional configuration, which extends axially and linearly along a side surface of the mounting portion 14c in continuation with the groove-forming portion 15a. Although one groove portion 15 may be formed, as shown in FIG. 7, it is preferable that the groove portion 15 is formed in a plural number in such a way that the groove portions 15 are equiangular with respect to the axis of the projected member 14 for breaking use. The number of the groove portions 15 is preferably 2 to 10. In the syringe 1, the groove portion 15 extends axially from the rear end of the projected portion 14b for breaking use to the front end of the mounting portion 14c, and as shown in FIGS. 2 and 7, three groove portions 15 are formed at intervals of about 120°. The configuration of the projected portion for breaking use is not limited to the above-described one, but the projected portion for breaking use is capable of taking any configurations, provided that the projected portion for breaking use penetrates through the sealing member readily.

The solid projected member for breaking use may have a construction as shown in FIG. 8. A projected member for breaking use 40 shown in FIG. 8 is mounted on the inner open portion 29 of the needle-mounting portion 21 of the outer cylinder 2 in such a way that the projected member for breaking use 40 extends to the rear-end side of the outer cylinder 2. The projected member for breaking use 40 has a projected portion for breaking use 40b extending toward the rear-end side of the outer cylinder 2, a mounting portion 40c to be mounted on the outer cylinder 2, and a liquid medicine guide path 41 for guiding the liquid medicine from the side of the projected portion for breaking use 40b to the side of the needle-mounting portion 21. The projected portion for breaking use 40b projects from the neighborhood of the needle-mounting portion 21 of the outer cylinder 2 toward the rear-end side of the outer cylinder 2 and has a sealing member-breaking blade portion 40d at the rear end thereof. The projected member for breaking use 40 is fixed to the outer cylinder 2 by fitting the mounting portion 40c into the inner open portion 29 of the needle-mounting portion 21.

More specifically, the projected member for breaking use 40 is made of a material different from that of the outer cylinder 2 and is solid. The projected member for breaking use 40 has a disk-shaped body portion 40a; the needle-shaped projected portion for breaking use 40b disposed at the rear-end side (upper side in FIG. 8) of the body portion 40a in such a way that the needle-shaped projected portion for breaking use 40b is formed in the shape of a cylinder having almost same diameter at any portions thereof except a sealing member-breaking blade portion 40d formed in the shape of a slope at the rear end of the projected member for breaking use 40; the rod-shaped mounting portion 40c disposed at the front-end side (lower side in FIG. 8) of the body portion 40a in such a way that the diameter thereof decreases taperingly toward the front-end side of the outer cylinder 2; and a groove portion (liquid medicine guide path) 41 extending axially from the rear end of the body portion 40a to the front end of the mounting portion 40c along a side surface of the projected member for breaking use 40.

The groove portion 41 extends axially and linearly along the side surface of the projected member for breaking use 40 from the rear end of the body portion 40a to the front end of the mounting portion 40c. The bottom surface of the groove portion 41 is curved outward. The groove portion 41 is formed to have the same width from the rear end of the body portion 40a to the front end of the mounting portion 40c. In this embodiment, one groove portion 41 is formed. The groove portion 41 may be formed in a plural number. When a plurality of the groove portions 41 is formed, it is preferable that they are formed equiangularly with respect to the axis of the projected member for breaking use. The configuration of the sealing member-breaking blade portion is not limited to the above-described one, but may have any desired configurations, provided that the projected member for breaking use penetrates through the sealing member readily.

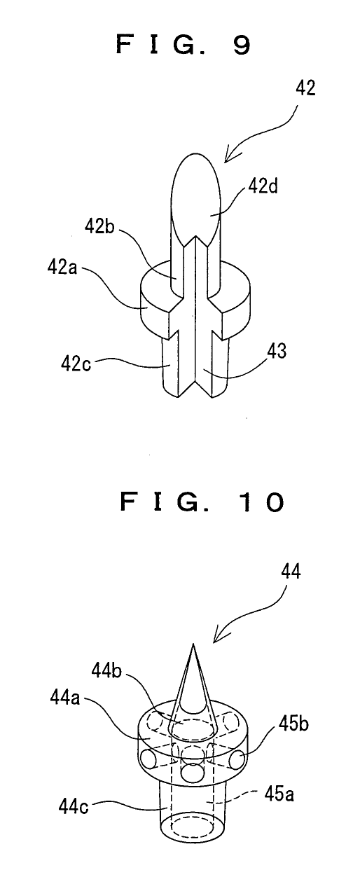

The solid projected member for breaking use may have a construction as shown in FIG. 9. A projected member for breaking use 42 shown in FIG. 9 is mounted on the inner open portion 29 of the needle-mounting portion 21 of the outer cylinder 2 in such a way that the projected member for breaking use 42 extends to the rear-end side of the outer cylinder 2. The projected member for breaking use 42 has a projected portion for breaking use 42b extending to the rear-end side of the outer cylinder 2, a mounting portion 42c to be mounted on the outer cylinder 2, and a liquid medicine guide path for guiding the liquid medicine from the side of the projected portion for breaking use 42b to the side of the needle-mounting portion 21. The projected portion for breaking use 42b projects from the neighborhood of the needle-mounting portion 21 of the outer cylinder 2 toward the rear-end side of the outer cylinder 2 and has a sealing member-breaking blade portion 42d at the rear end thereof. The projected member for breaking use 42 is fixed to the outer cylinder 2 by fitting the mounting portion 42c into the inner open portion 29 of the needle-mounting portion 21.

More specifically, the projected member for breaking use 42 is made of a material different from that of the outer cylinder 2 and is solid. The projected member for breaking use 42 has a disk-shaped body portion 42a; the projected portion for breaking use 42b disposed at the rear-end side (upper side in FIG. 9) of the body portion 42a in such a way that the projected portion for breaking use 42b is formed in the shape of a cylinder having almost same diameter at any portions thereof except a blade portion 42d formed in the shape of a slope; the rod-shaped mounting portion 42c disposed at the front-end side (lower side in FIG. 9) of the body portion 42a in such a way that the diameter thereof decreases taperingly toward the front-end side of the outer cylinder 2; and a groove portion 43 extending axially from the neighborhood of a front-end side portion of the blade portion 42d to the front end of the mounting portion 42c along a side surface of the projected member for breaking use 42.

Along a side surface of the projected member for breaking use 42, the groove portion 43 extends axially and linearly from the neighborhood of the front-end side portion of the blade portion 42d formed in the shape of a slope to the front end of the mounting portion 42c. The sectional configuration of the groove portion is triangular. In this embodiment, one groove portion 43 is formed. The groove portion 43 may be formed in a plural number. When a plurality of the groove portions 43 is formed, it is preferable that they are formed equiangularly with respect to the axis of the projected member for breaking use. The configuration of the blade portion of the projected portion for breaking use 42b is not limited to the above-described one, but the blade portion of the projected portion for breaking use 42b may be configured as desired, provided that the projected portion for breaking use 42b penetrates through the sealing member readily.

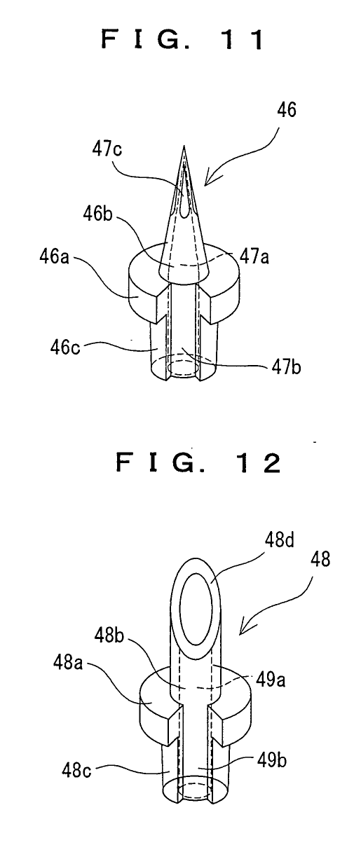

The projected member for breaking use may be formed hollowly, although it is solid in the above description. As shown in FIG. 10, the hollow projected member for breaking use 44 is mounted on the inner open portion 29 of the needle-mounting portion 21 of the outer cylinder 2 in such a way that the hollow projected member for breaking use 44 extends to the rear-end side of the outer cylinder 2. The projected member for breaking use 44 has a projected portion for breaking use 44b extending to the rear-end side of the outer cylinder 2, a mounting portion 44c to be mounted on the outer cylinder 2, and liquid medicine guide paths 45a, 45b for guiding the liquid medicine from the side of the projected portion for breaking use 44b to the side of the needle-mounting portion 21. The projected portion for breaking use 44b projects from the neighborhood of the needle-mounting portion 21 of the outer cylinder 2 toward the rear-end side of the outer cylinder 2 and has a sealing member-breaking pointed-end portion at the rear end thereof. The projected member for breaking use 44 is fixed to the outer cylinder 2 by fitting the mounting portion 44c into the inner open portion 29 of the needle-mounting portion 21.

More specifically, as shown in FIG. 10, the hollow projected member for breaking use 44 is made of a material different from that of the outer cylinder 2. The projected member for breaking use 44 has a disk-shaped body portion 44a; the needle-shaped projected portion for breaking use (projected portion for breaking use having the sealing member-breaking pointed-end portion) 44b disposed at the rear-end side (upper side in FIG. 10) of the body portion 44a; the rod-shaped mounting portion 44c disposed at the front-end side (lower side in FIG. 10) of the body portion 44a in such a way that the diameter thereof decreases taperingly toward the front-end side of the outer cylinder 2; a main path (liquid medicine guide path) 44a which is open at the rear-end portion of the projected portion for breaking use 44b and at the front-end surface of the mounting portion 44c and extends inside the projected member for breaking use 44 from the rear-end portion of the projected portion for breaking use 44b to the front end of the mounting portion 44c; and an auxiliary path (liquid medicine guide path) 45b open on a side surface of the body portion and communicating with the main path 45a from the side surface of the body portion inside the projected member for breaking use 44. It is preferable to form a plurality of auxiliary paths 45b in such a way that they are equiangular with respect to the axis of the projected member for breaking use 44. The number of the auxiliary paths 45b is preferably 2 to 10. In the projected member 44 for breaking use shown in FIG. 10, six auxiliary paths 45b are formed equiangularly at intervals of about 60 degrees with respect to the main path 45a.

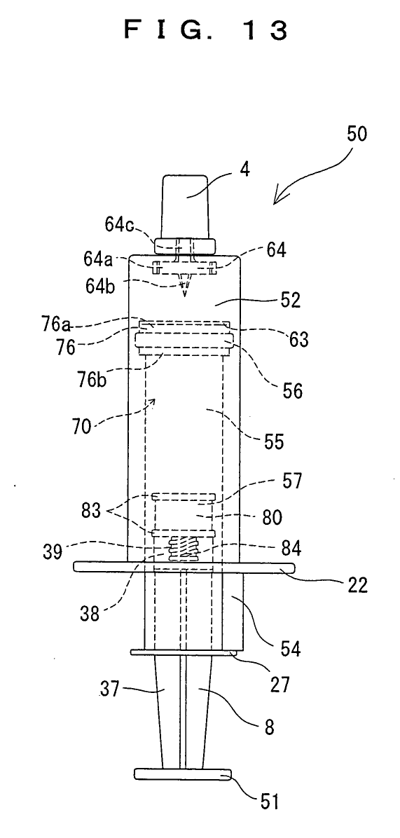

The hollow projected member for breaking use may have a construction, as shown in FIG. 11. The hollow projected member for breaking use 46 is mounted on the inner open portion 29 of the needle-mounting portion 21 of the outer cylinder 2 in such a way that the projected member for breaking use 46 extends to the rear-end side of the outer cylinder 2. The projected member for breaking use 44 has a projected portion for breaking use 46b extending to the rear-end side of the outer cylinder 2, a mounting portion 46c to be mounted on the outer cylinder 2, and liquid medicine guide paths 47a, 47b for guiding the liquid medicine from the side of the projected portion for breaking use 46b to the side of the needle-mounting portion 21. The projected portion for breaking use 46b projects from the neighborhood of the needle-mounting portion 21 of the outer cylinder 2 toward the rear-end side of the outer cylinder 2 and has a sealing member-breaking pointed-end portion at the rear end thereof. The projected member for breaking use 46 is fixed to the outer cylinder 2 by fitting the mounting portion 46c into the inner open portion 29 of the needle-mounting portion 21.

More specifically, as shown in FIG. 11, the hollow projected member for breaking use 46 is made of a material different from that of the outer cylinder 2. The projected member for breaking use 46 has a disk-shaped body portion 46a; the needle-shaped projected portion for breaking use (projected portion for breaking use having the sealing member-breaking pointed-end portion) 46b disposed at the rear-end side (upper side in FIG. 11) of the body portion 46a; the rod-shaped mounting portion 46c disposed at the front-end side (lower side in FIG. 11) of the body portion 46a in such a way that the diameter thereof decreases taperingly toward the front-end side of the outer cylinder 2; a liquid medicine guide hole (liquid medicine guide path) 47a formed inside the projected member for breaking use 46; and a groove portion (liquid medicine guide path) 47b formed on a side surface of the projected member for breaking use 46.

The liquid medicine guide hole 47a extends axially from the rear-end portion of the projected portion for breaking use 46b to the front end of the mounting portion 46c inside the projected member for breaking use 46 and is open at the rear-end portion of the projected portion for breaking use 46b as well as on the front-end surface of the mounting portion 46c. A plurality of open portions 47c is formed at the rear-end portion of the projected portion for breaking use 46b. One open portion is formed on the front-end surface of the mounting portion 46c. It is preferable that 1 to 8 open portions are formed at the rear-end portion of the projected portion for breaking use 46b. The open portions 47c are disposed equiangularly with respect to the axis of the projected portion for breaking use 46b. In this embodiment, the open portion 47c is spindle-shaped. Four open portions 47c are formed at intervals of 90 degrees. The configuration of the open portion at the rear-end portion is not limited to this but may be approximately elliptic.

The groove portion 47b extends axially and linearly along the side surface of the projected member for breaking use 46 from the rear end of the body portion 46a to the front end of the mounting portion 46c. The bottom surface of the groove portion 47b is curved outward. The groove portion 47 is formed to have the same width from the rear end of the body portion 46a to the front end of the mounting portion 46c. In this embodiment, one groove portion 47 is formed. The groove portion 47 may be formed in a plural number. When a plurality of the groove portions 47 is formed, it is preferable that they are formed equiangularly with respect to the axis of the projected member for breaking use. The configuration of the pointed-end portion of the projected portion for breaking use 46b is not limited to the above-described one, but may have any desired configurations, provided that the projected portion for breaking use 46b penetrates through the sealing member readily.

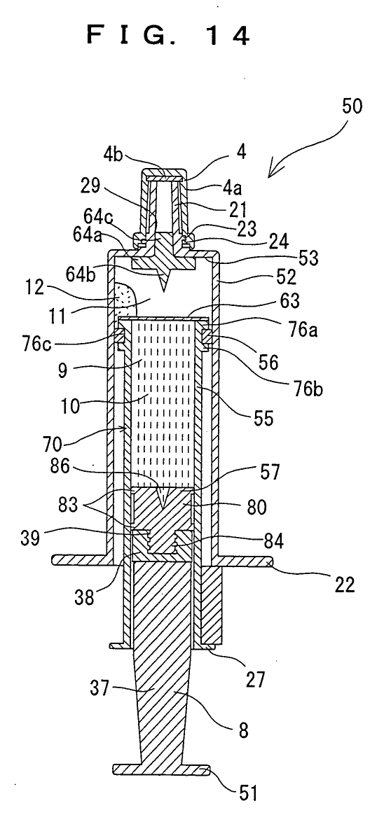

The hollow solid projected member for breaking use have a construction as shown in FIG. 12. The hollow projected member for breaking use 48 is mounted on the inner open portion 29 of the needle-mounting portion 21 of the outer cylinder 2 in such a way that the projected member for breaking use 48 extends to the rear-end side of the outer cylinder 2. The projected member for breaking use 48 has a projected portion for breaking use 48b extending to the rear-end side of the outer cylinder 2, a mounting portion 48c to be mounted on the outer cylinder 2, and liquid medicine guide paths 49a, 49b for guiding the liquid medicine from the side of the projected portion for breaking use 48b to the side of the needle-mounting portion 21. The projected portion for breaking use 48b projects from the neighborhood of the needle-mounting portion 21 of the outer cylinder 2 toward the rear-end side of the outer cylinder 2 and has a sealing member-breaking blade portion at the rear end thereof. The projected member for breaking use 48 is fixed to the outer cylinder 2 by fitting the mounting portion 48c into the inner open portion 29 of the needle-mounting portion 21.

More specifically, the projected member for breaking use 48 is made of a material different from that of the outer cylinder 2 and is hollow. The projected member for breaking use 48 has a disk-shaped body portion 48a; the projected portion for breaking use 48b disposed at the rear-end side (upper side in FIG. 12) of the body portion 48a in such a way that the projected portion for breaking use 48b is formed in the shape of a cylinder having almost same diameter at any desired portions thereof except a blade portion (rear-end surface) 48d formed in the shape of a slope; the rod-shaped mounting portion 48c disposed at the front-end side (lower side in FIG. 12) of the body portion 48a in such a way that the diameter thereof decreases taperingly toward the front-end side of the outer cylinder 2; the liquid medicine guide hole (liquid medicine guide path) 49a formed inside the projected member for breaking use 48; and the groove portion 49b formed on the side surface of the projected member for breaking use 48. Inside the projected member for breaking use 48, the liquid medicine guide hole 49a extends axially in an equal inner diameter from the rear-end surface of the projected portion for breaking use 48b to the front end of the mounting portion 48c and is open on the rear-end surface of the projected portion for breaking use 48b and on the front-end surface of the mounting portion 48c.

The groove portion 49b extends axially and linearly along the side surface of the projected member for breaking use 48 from the rear end of the body portion 48a to the front end of the mounting portion 48c. The bottom surface of the groove portion 49b is curved outward. The groove portion 49 has the same width from the rear-end surface of the body portion 48a to the front end of the mounting portion 48c. In this embodiment, one groove portion 49 is formed. The groove portion 49 may be formed in a plural number. When a plurality of the groove portions 49 is formed, it is preferable that they are formed equiangularly with respect to the axis of the projected member for breaking use. The configuration of the blade portion of the projected portion for breaking use 48b is not limited to the above-described one, but may have any desired configurations, provided that the projected portion for breaking use 48b penetrates through the sealing member readily.

The diameter of the disk-shaped body portion of the projected member for breaking use may decrease toward the projected portion for breaking use. In this case, the first gasket 6 which will be described later is so formed as to decrease the through-hole thereof toward its rear end. This construction allows the body portion of the projected member for breaking use to be easily inserted into the first gasket 6.

As described above, the projected portion for breaking use may be integral with the outer cylinder instead of making it of a material different from that of the outer cylinder.

As shown in FIGS. 1 and 2, the inner cylinder assemblage 20 has the inner cylinder 5, the first gasket 6, and the sealing member 13.

As shown in FIGS. 1 and 2, the inner cylinder 5 is cylindrical and its front end and rear end are open. At the front-end portion of the inner cylinder 5, there is formed a gasket-mounting portion 26 decreasing in its diameter toward its front-end side and having an enlarged diameter at its front end. An annular flange 27 is formed at the rear end of the inner cylinder 5. A front end opening of the inner cylinder 5 is airtightly closed with the sealing member 13.

As the constituent material for the inner cylinder 5, it is preferable to use a constituent material similar to that of the outer cylinder 2. As the constituent material for the inner cylinder 5, it is preferable to use a material having a low vapor permeability so that the medicine-dissolving liquid 10 inside the first accommodation portion 9 does not prevent maintenance of a dry state of the medicine 12 inside the second accommodation portion 11. It is preferable that the length of the inner cylinder 5 is 5 to 200 mm. It is preferable that the volume of the inner cylinder 5 is 1 to 60 ml.

The sealing member 13 is formed circularly and thinly to airtightly seal the front end opening of the inner cylinder 5.

The sealing member 13 has the property of being vapor-unpermeable therethrough or the property of being hard for vapor to permeate therethrough. More specifically, the sealing member 13 has a covering layer consisting of a vapor-unpermeable material or a material making it difficult for vapor to permeate therethrough. As the vapor-unpermeable material, it is preferable to use aluminum, aluminum oxide, silicon oxide, and the like. It is preferable that the sealing member 13 has a synthetic resinous layer to prevent the sealing member 13 from scattering, when the sealing member 13 is broken by the projected portion 14b for breaking use. As the synthetic resin for use in the synthetic resinous layer, polyethylene, polypropylene, polyester, and the like are preferable. As the sealing member 13, it is preferable to use a multi-layer film consisting of polyethylene, polypropylene or polyester over one side of which or both sides of which the vapor-unpermeable material or the material making it difficult for vapor to permeate therethrough such as aluminum, aluminum oxide, silicon oxide or the like is evaporated or layered. It is particularly preferable to use a resinous film consisting of polyethylene, polypropylene or polyester over one side of which aluminum oxide is evaporated or aluminum foils are layered. It is preferable to evaporate aluminum, aluminum oxide, silicon oxide or the like over the film by a CVD method.

The thickness of each of the aluminum oxides evaporated over both sides of the resinous film is preferably 0.01 to 0.1 μm. The thickness of the resinous film is preferably 10 to 100 μm.

It is preferable that the sealing member 13 has an adhesive layer to fix the sealing member 13 to the inner cylinder 5. As the adhesive layer, a hot-melt adhesive layer such as low-melting polyethylene can be used. The sealing member 13 is fixed to the front end opening of the inner cylinder 5 by means of thermal fusing, high-frequency fusing or ultrasonic wave fusing. When the sealing member 13 does not have an adhesive layer, the sealing member 13 may be bonded to the inner cylinder 5 by using an instantaneous adhesive agent such as cyanoacrylate and an UV-curing adhesive agent.

The sealing member is thin in the embodiment of the present invention. But provided that the projected portion for breaking use is capable of penetrating through the sealing member, the sealing member may be thick to some extent.

The first gasket 6 is annular and liquid-tightly slidable in contact with the inner wall of the outer cylinder and has a through-hole 17 allowing the projected portion 14b for breaking use to enter therethrough. The first gasket 6 is mounted on the gasket-mounting portion (front-end portion) 26 of the inner cylinder 5, with the first gasket 6 surrounding the peripheral portion of the sealing member 13. The first gasket 6 has a body portion 16 extending in almost the same diameter, a front-end portion 18 disposed at the front end of the body portion 16 in such a way that the front-end portion 18 taperingly decreases in its diameter toward the front-end side of the outer cylinder 2, and two annular ribs 19 liquid-tightly contacting the outer cylinder 2 disposed on the peripheral surface of the body portion. It is preferable that the inner surface of the first gasket 6 is so configured as to closely liquid-tightly and airtightly contact the front-end portion of the inner cylinder 5. For example, it is preferable to form the inner diameter of the first gasket 6 a little smaller than the outer diameter of the inner cylinder 5 at its front-end portion so that the first gasket 6 contacts the inner cylinder 5 closely. More specifically, it is preferable that the inner surface of the first gasket 6 has a configuration of sandwiching the gasket-mounting portion 26 of the inner cylinder 5. By forming the first gasket 6 in this manner, after the sealing member 13 is broken by the projected portion 14b for breaking use, the liquid medicine is prevented from penetrating into a space between the outer cylinder 2 and the inner cylinder 5. The above-described annular first gasket 6 may be formed in the shape of a cylinder having a length to some extent or in the shape of a ring or the like axially short as will be described later.

The front-end side portion (side forward from the sealing member 13) of the through-hole 17 extends in almost the same inner diameter. As shown in FIG. 6, when the inner cylinder assemblage 20 moves to the front-end side, the projected portion 14b for breaking use is capable of passing through the through-hole 17 and accommodating the body portion 14a of the projected member 14 for breaking use therein. The rear-end side inner surface of the through-hole 17 has a configuration allowing the gasket-mounting portion 26 of the inner cylinder 5 to fit on the through-hole 17. The configuration of a tapered surface of the front-end portion of the first gasket 6 corresponds to that of the inner surface of the front-end side of the outer cylinder 2 to prevent a gap from being generated therebetween as much as possible, when the front-end portion of the first gasket 6 contacts an inner surface 28 of the front end of the outer cylinder 2. The peripheral portion of the sealing member 13 is covered with the first gasket 6. The central portion of the sealing member 13 is exposed inside the through-hole 17 and can be penetrated by the projected portion 14b for breaking use. The gasket 6 does not necessarily have the through-hole 17, provided that the material for the gasket 6 and the thickness thereof allow penetration of the projected portion 14b for breaking use through the sealing member 13.

As the constituent material for the first gasket 6, it is possible to use elastic rubber (for example, butyl rubber, latex rubber, silicone rubber, and the like); synthetic resin (SBS elastomer, SEBS elastomer, SEPS elastomer, SIS elastomer, polyolefin elastomer, and the like); and rubber or synthetic resin covered with a film such as PTFE, ETFE, FEP, ultra-high-density polyethylene, and the like.

The second gasket 7 is liquid-tightly slidable in contact with the inner wall of the inner cylinder and has a plunger-mounting portion at its rear-end portion. More specifically, as shown in FIGS. 1 and 2, the second gasket 7 has a body portion 30 extending in an almost equal outer diameter, a tapered portion disposed at the front end of the body portion 30 and decreasing taperingly in its diameter toward the front-end side, a front-end portion 31 having a projected portion 32 disposed at the front-end side of the tapered portion and having almost an equal outer diameter, two annular ribs 33 liquid-tightly contacting the inner cylinder 5 provided on the peripheral surface of the body portion, and a male screw portion 34, provided at the rear end of the body portion 30, which engages the plunger 8. The configuration of the front-end surface of the second gasket 7 corresponds to that of the inner surface of the front-end side of the inner cylinder 5 to prevent a gap from being generated therebetween as much as possible.

The second gasket 7 has an accommodation portion 36 which is formed in the direction from the front end thereof to the rear-end side and capable of accommodating the projected portion for breaking use partly or entirely. As shown in FIGS. 1 and 6, the accommodation portion 36 is formed as a conic concavity capable of accommodating almost the entire projected portion 14b for breaking use of the projected member 14 for breaking use. It is preferable that the accommodation portion has a configuration which allows accommodation of the projected portion for breaking use without a gap. By forming the accommodation portion 36 in the second gasket 7, it is possible to reduce formation of a dead space when the plunger 8 is pressed completely to the front end and hence reduce a residual amount of the liquid medicine. Although in the embodiment of the present invention, the accommodation portion 36 has a configuration which allows accommodation of only the projected portion 14b for breaking use, the accommodation portion 36 may have a configuration which allows accommodation of other portions of the projected member for breaking use. It is preferable to use a material similar to that of the first gasket 6 as the constituent material of the second gasket 7. When the material of the second gasket is flexible enough for the projected portion for breaking use to pierce thereinto, it is unnecessary to provide the second gasket with the accommodation portion.

The plunger 8 has a body portion 37 cross-shaped in section and extending axially; a front-end portion 38 having a female screw portion 39 engaging a male screw portion 34 formed at the rear-end portion of the second gasket 7; and a disk-shaped portion 51, for pressing use, disposed at the rear end of the body portion 37. The front-end portion 38 is columnar and has the female screw portion 39 (concavity) therein.

The two-chamber type pre-filled syringe 1 has the first accommodation portion 9 formed among the rear portion of the sealing member 13, the front portion of the second gasket 7, and the inner peripheral surface of the inner cylinder; the medicine-dissolving liquid 10 accommodated in the first accommodation portion 9; the second accommodation portion 11 formed among the front portion of the first gasket 6, the front portion of the sealing member 13 (inner cylinder assemblage 20), and the inner peripheral surface of the outer cylinder 2; the powdery or frozen dry medicine 12 accommodated inside the second accommodation portion 11. It is preferable that the volume of the first accommodation portion 9 is 1 to 20 ml, although it depends on the amount of the medicine to be accommodated therein. It is preferable that the volume of the second accommodation portion 11 is 2 to 25 ml, although it depends on the amount of the medicine-dissolving liquid to be accommodated therein.

As the powdery or frozen dry medicine 12 accommodated in the first accommodation portion 9, medicines such as vitamins, an antibiotic, a vasodilator, a cardiotonic drug, and the like and a medicine for promoting nutrition are used.

As the medicine-dissolving liquid 10, distilled water for injection and physiological saline are used. The second accommodation portion 11 may be formed as a decompressed space. By doing so, a liquid injection work can be facilitated. In this case, it is necessary to provide an inner cylinder stopping mechanism 54 for preventing the inner cylinder 5 from moving toward the front end of the outer cylinder 2. The inner cylinder stopping mechanism 54 is a plate-shaped member contacting the rear-end surface of the flange 22 of the outer cylinder 2 and the front-end surface of the flange 27 of the inner cylinder 5. Unless the inner cylinder stopping mechanism 54 is removed, the inner cylinder 5 does not move to the front end of the outer cylinder 2.

The operation of the above-described two-chamber type pre-filled syringe 1 is described below with reference to FIGS. 2 through 6.

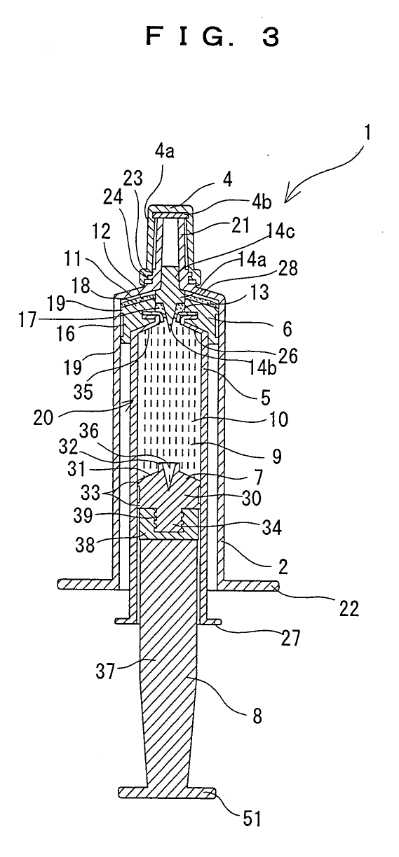

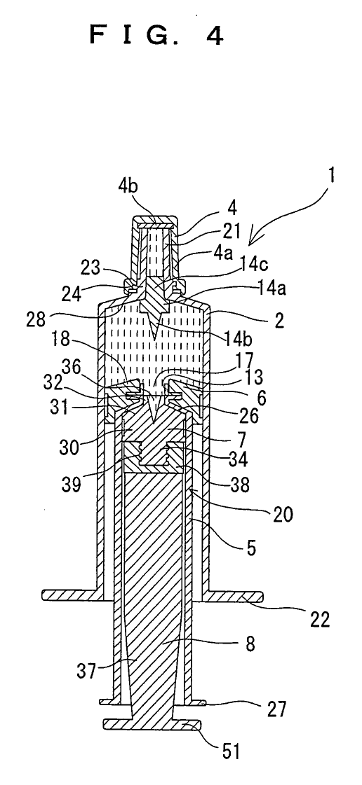

An inner cylinder stopping mechanism 54 is removed from the two-chamber type pre-filled syringe 1 placed in a state shown in FIG. 2. When the plunger 8 is gradually pressed toward the front-end side of the outer cylinder 2, the inner cylinder assemblage 20 moves toward the front-end side of the outer cylinder 2, and the rear end of the projected portion 14b for breaking use breaks the sealing member 13 of the first gasket 6, as shown in FIG. 3. As a result, the first accommodation portion 9 and the second accommodation portion 11 communicate with each other. By further pressing the plunger 8 toward the front-end side of the outer cylinder, the medicine-dissolving liquid 10 accommodated in the first accommodation portion 9 shifts into the second accommodation portion 11, and the first gasket 6 moves to the rear-end side of the outer cylinder. Consequently the two-chamber type pre-filled syringe 1 has a state of FIG. 4. Then the syringe 1 is shaken to securely dissolve the medicine in the medicine-dissolving liquid 10. Thereafter the sealing cap 4 is removed from the needle-mounting portion 21, and then as shown in FIG. 5, the needle 25 is mounted on the needle-mounting portion 21 of the outer cylinder 2. Thereby preparation for administering the liquid medicine is completed.

Then the cap 25b of the needle 25 is removed. After air inside the syringe 1 is eliminated, the syringe 1 is pierced into a necessary portion of a patient. By pressing the plunger 8, the liquid medicine inside the syringe 1 is administered to the patient. In a state where the plunger 8 is pressed completely toward the front-end side of the outer cylinder, as shown in FIG. 6, the projected portion 14b for breaking use is accommodated in the accommodation portion 36 of the second gasket 7, and the body portion 14a of the projected member 14 for breaking use is accommodated inside the through-hole 17 of the first gasket 6 without a gap. Therefore the amount of the liquid medicine which remains inside the syringe 1 is very small. When the sealing cap is of a type in which the double ended needle is mounted on the needle-mounting portion, a medicine may be administered with the double ended needle mounted on the sealing cap without removing the sealing cap from the needle-mounting portion.

The two-chamber type pre-filled syringe of another embodiment of the present invention will be described below.

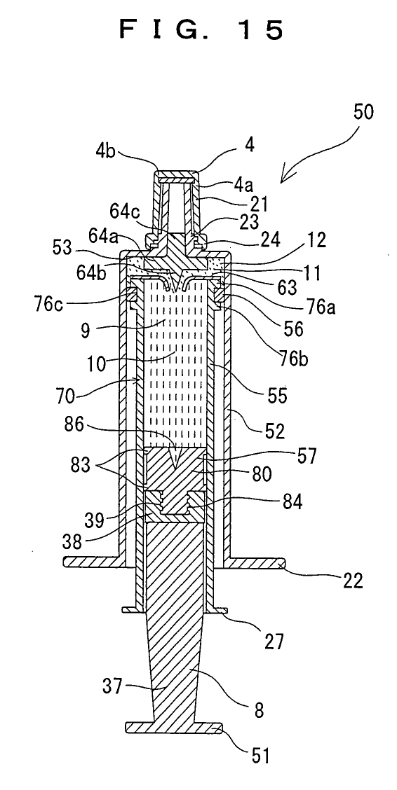

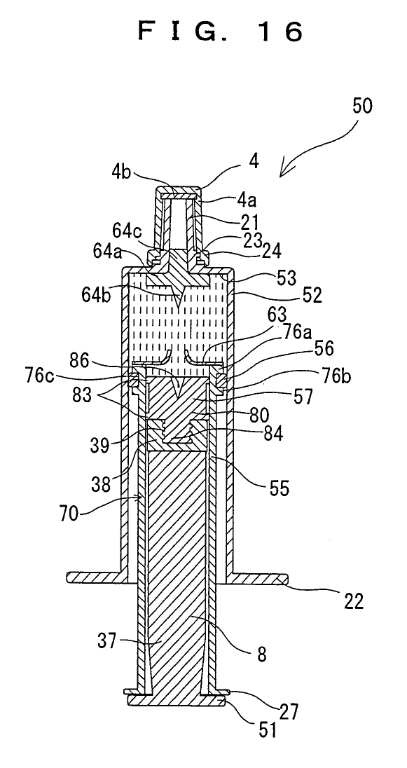

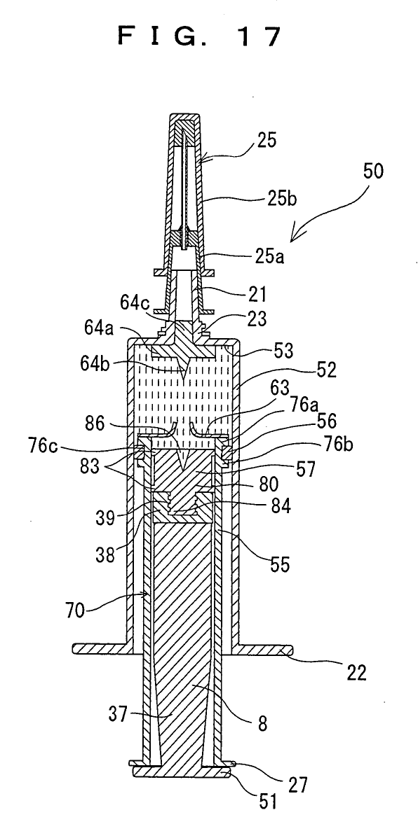

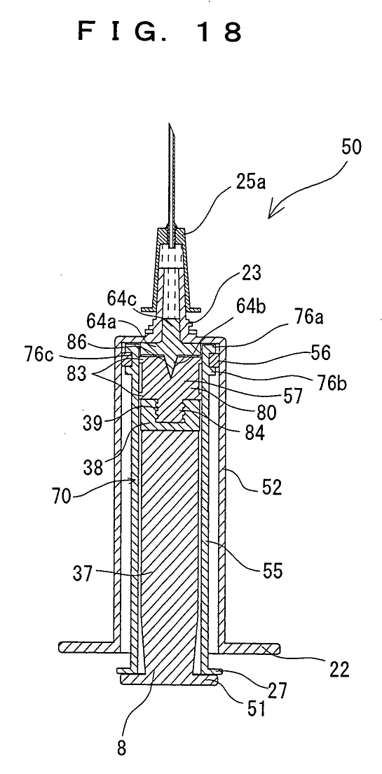

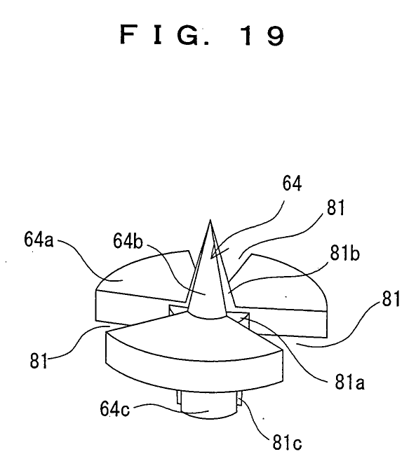

FIG. 13 is an outlook view of a two-chamber type pre-filled syringe according to another embodiment of the present invention. FIG. 14 is a sectional view of the two-chamber type pre-filled syringe shown in FIG. 13. FIG. 15 is an explanatory view for explaining the operation of the two-chamber type pre-filled syringe shown in FIG. 13. FIG. 16 is an explanatory view for explaining the operation of the two-chamber type pre-filled syringe shown in FIG. 13. FIG. 17 is an explanatory view for explaining the operation of the two-chamber type pre-filled syringe shown in FIG. 13. FIG. 18 is an explanatory view for explaining the operation of the two-chamber type pre-filled syringe shown in FIG. 13. FIG. 19 is a perspective view of a projected portion for breaking use which is used in the two-chamber type pre-filled syringe shown in FIG. 13.

A pre-filled syringe 50 of the present invention includes an outer cylinder 52 having a needle-mounting portion 21 and a projected portion 64b for breaking use disposed inside the outer cylinder 52 and projecting toward a rear end thereof; closing members 4, 25 mounted on the needle-mounting portion 21; an inner cylinder assemblage 70 having a cylindrical inner cylinder 55 accommodated inside the outer cylinder 52 and being open at both ends thereof, an annular first gasket 56 provided in the vicinity of a front-end portion of the inner cylinder 55 and slidable inside the outer cylinder 52, and a sealing member 63 which airtightly seals a front-end portion of the inner cylinder 55 and can be broken by the projected portion 64b for breaking use provided inside the outer cylinder 52; a second gasket 57 sidably accommodated inside the inner cylinder 55; a plunger 8 which is mounted or can be mounted at a rear-end portion of the second gasket 57; a first accommodation portion 9 formed among the sealing member 63, the second gasket 57, and the inner cylinder 55; a second accommodation portion 11 formed between the inner cylinder assemblage 70 and the outer cylinder 52; a medicine-dissolving liquid 10 accommodated in the first accommodation portion 9; and a powdery or frozen dry medicine 12 accommodated inside the second accommodation portion 11. The main difference between the two-chamber type pre-filled syringe 50 of this embodiment and the above-described two-chamber type pre-filled syringe 1 is that the first gasket 6 of the two-chamber type pre-filled syringe 1 is axially long to some extent and annular, whereas the first gasket 56 is axially short. The outer cylinder 52 is the same as the outer cylinder 2 of the two-chamber type pre-filled syringe 1 described in the above embodiment. The closing members 4 and 25 are the same as that of the two-chamber type pre-filled syringe 1 described in the above embodiment.

In this embodiment, the two-chamber type pre-filled syringe 50 has also a projected member 64 for breaking use mounted on the inner open portion 29 of the needle-mounting portion 21 disposed inside the outer cylinder 52. As shown in FIG. 19, the projected member 64 for breaking use has a projected portion 64b for breaking use extending toward the rear-end side of the outer cylinder 52, a mounting portion 64c to be mounted on the outer cylinder 52, and a medicine guide path 81 for guiding the liquid medicine from the side of the projected portion 64b for breaking use to the side of the needle-mounting portion 21. The projected member 64 for breaking use projects from the neighborhood of the inner open portion 29 of the needle-mounting portion 21 of the outer cylinder 52 toward the rear-end side of the outer cylinder and has a sealing member-breaking pointed-end portion disposed at the rear end thereof.

More specifically, the projected member 64 for breaking use has a disk-shaped body portion 64a; the needle-shaped projected portion 64b for breaking use (having the sealing member-breaking pointed-end portion disposed at the rear end thereof) disposed at the rear end of the body portion 64a; and the rod-shaped mounting portion 64c disposed at the front end of the body portion 64a in such a way that the diameter thereof decreases taperingly toward the front-end side. The basic construction of the projected member 64 for breaking use is the same as that of the above-described projected member 14 for breaking use. The difference between both is that the diameter of the disk-shaped body portion 64a of the projected member 64 for breaking use is larger than the diameter of the disk-shaped body portion of the projected member 14 for breaking use. The projected member 64 for breaking use is fixed to the outer cylinder by fitting the mounting portion 64c into the inner open portion 29 of the needle-mounting portion 21.

The groove portion 81 has a groove-forming portion 81b which is triangular in its sectional configuration and extends axially and linearly on the side surface of the projected portion 64b for breaking use; a groove-forming portion 81a formed continuously with the groove-forming portion 81b along the upper, side, and lower surfaces of the disk-shaped body portion 64a; and a groove-forming portion 81c, triangular in its sectional configuration, which extends axially and linearly in continuation with the groove-forming portion 64c on the side surface of the mounting portion 64c. Although one groove portion 81 may be formed, it is preferable that as embodied in the present invention, the groove portion 81 is formed in a plural number in such a way that the groove portions 81 are equiangular with respect to the axis of the projected member 64 for breaking use. The number of the groove portions 81 is preferably 2 to 10. In the syringe 50 of this embodiment, the groove portion 81 extends axially from the rear end of the projected portion 64b for breaking use to the front end of the mounting portion 64c, and three groove portions 81 are formed at intervals of about 120°. In the syringe of this embodiment, the projected portion 64b for breaking use is needle-shaped, but the projected portion 64b for breaking use can be formed in any desired configurations, provided that the projected portion 64b for breaking use penetrates through the sealing member.

As shown in FIGS. 13 and 14, the inner cylinder assemblage 70 has the inner cylinder 55, the first gasket 56, and the sealing member 63.

As shown in FIG. 14, the inner cylinder 55 is open at its front and rear ends and has entirely almost the same diameter. At the front-end portion of the inner cylinder 55, a gasket-mounting portion 76 on which the first gasket 56 is mounted is disposed. At the rear end of the inner cylinder 55, a flange 27 is disposed. A front end opening of the inner cylinder 55 is airtightly closed with the sealing member 63. As the constituent material for the inner cylinder 55, it is preferable to use a constituent material similar to that of the inner cylinder 5. It is preferable that the length of the inner cylinder 55 is 5 to 200 mm. It is preferable that the volume of the inner cylinder 55 is 1 to 60 ml.

The gasket-mounting portion 76 is formed as an annular concavity 76c constructed of an annular convexity 76a disposed at the front end of the inner cylinder 55, an annular convexity 76b formed rearward from the annular convexity 76a, and the peripheral surface of the inner cylinder. By fitting the annular first gasket 56 on the annular concavity 76c, the first gasket 56 is mounted on the inner cylinder 55. The gasket-mounting portion may be formed as an annular concavity formed on the peripheral surface of the front-end portion of the inner cylinder.

The first gasket 56 is liquid-tightly slidable in contact with the inner wall of the outer cylinder. As shown in FIGS. 13 and 14, the first gasket 56 is ring-shaped and mounted on the gasket-mounting portion 76 formed at the front-end portion of the inner cylinder 55. The constituent material for the first gasket 56 is similar to that of the first gasket 6.

The sealing member 63 is formed circularly and thinly to airtightly seal the front end opening of the inner cylinder 55. In the syringe of this embodiment, the sealing member 63 is entirely exposed. The sealing member 63 is fused onto the front end opening of the inner cylinder 5 by means of thermal fusing, high-frequency fusing or ultrasonic wave fusing. The sealing member 63 is formed in a manner similar to that of forming the sealing member 13. In the embodiments of the present invention, the sealing member is thinly formed but may be formed thickly to some extent, provided that the projected portion for breaking use is capable of penetrating through the sealing member.

The second gasket 57 is liquid-tightly slidable in contact with the inner wall of the inner cylinder. As shown in FIGS. 13 and 14, the second gasket 57 has a body portion 80 extending in an equal outer diameter, two annular ribs 83 liquid-tightly contacting the inner cylinder 55 provided on the peripheral surface of the body portion 80, and a male screw portion 84, disposed at the rear end of the body portion 80, which engages the plunger 8. It is preferable to use the constituent material for the second gasket 57 similar to that of the first gasket 56.

The second gasket 57 has an accommodation portion 86 which is formed in the direction from the front end thereof to the rear-end side and capable of accommodating the projected portion for breaking use partly or entirely. As shown in FIGS. 14 and 18, the accommodation portion 86 is formed as a conic concavity capable of accommodating almost the entire projected portion 64b for breaking use of the projected member 64 for breaking use. It is preferable that the accommodation portion 86 is formed in a configuration that allows accommodation of the projected portion for breaking use (break-through needle). By forming the accommodation portion 86 in the second gasket 57, it is possible to reduce formation of a dead space when the plunger 8 is pressed completely to the front-end side and hence reduce a residual amount of the liquid medicine. It is preferable that the accommodation portion 86 of the second gasket 57 is capable of accommodating the projected member for breaking use partly or entirely without a gap. Although the accommodation portion 86 has a configuration allowing accommodation of only the projected portion 64b for breaking use in the embodiment of the present invention, the accommodation portion 86 may have a configuration allowing accommodation of other portions of the projected member for breaking use.

The construction of the plunger 8, the first accommodation portion 9, the second accommodation portion 11, the medicine-dissolving liquid 10, the medicine 12, and the inner cylinder stopping mechanism 54 are as described above.

The operation of the above-described two-chamber type pre-filled syringe 50 is described below with reference to FIGS. 14 through 18.

An inner cylinder stopping mechanism 54 is removed from the two-chamber type pre-filled syringe 50 placed in a state shown in FIG. 14. When the plunger 8 is gradually pressed toward the front-end side of the outer cylinder, the inner cylinder assemblage 70 moves toward the front-end side of the outer cylinder, and the rear end of the projected portion 64b for breaking use breaks through the sealing member 63 of the first gasket 56, as shown in FIG. 15. As a result, the first accommodation portion 9 and the second accommodation portion 11 communicate with each other. By further pressing the plunger 8 toward the front-end side of the outer cylinder, the medicine-dissolving liquid 10 accommodated in the first accommodation portion 9 shifts into the second accommodation portion 11, and the first gasket 56 moves to the rear-end side of the outer cylinder. Then the syringe 50 is shaken to securely dissolve the medicine in the medicine-dissolving liquid 10. Thereafter the sealing cap 4 is removed from the needle-mounting portion 21, and the needle 25 is mounted on the needle-mounting portion 21 of the outer cylinder 52. Thereby preparation for administering the liquid medicine is completed (FIG. 17).

Then the cap 25b of the needle 25 is removed. After air inside the syringe 50 is eliminated, the syringe 50 is pierced into a necessary portion of a patient. By pressing the plunger 8, the liquid medicine inside the syringe 50 is administered. In a state where the plunger 8 is pressed completely to the front-end side of the outer cylinder, as shown in FIG. 18, the projected portion 64b for breaking use is accommodated in the accommodation portion 86 of the second gasket 57, and the disk-shaped body portion 64a of the projected member 64 for breaking use is accommodated in the vicinity of the front-end portion of the inner cylinder 55 without a gap. Therefore almost all of the liquid medicine inside the syringe 50 can be discharged. When the sealing cap is of a type in which the double ended needle is mounted on the needle-mounting portion, a medicine may be administered with the double ended needle mounted on the sealing cap without removing the sealing cap from the needle-mounting portion.

The two-chamber type pre-filled syringe of another embodiment of the present invention will be described below.

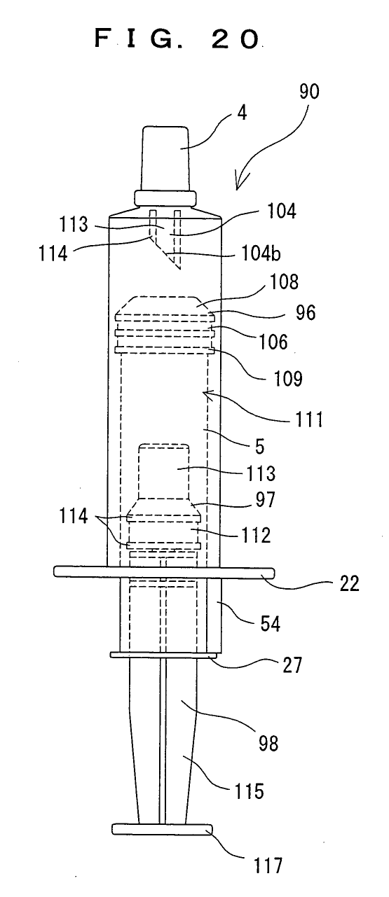

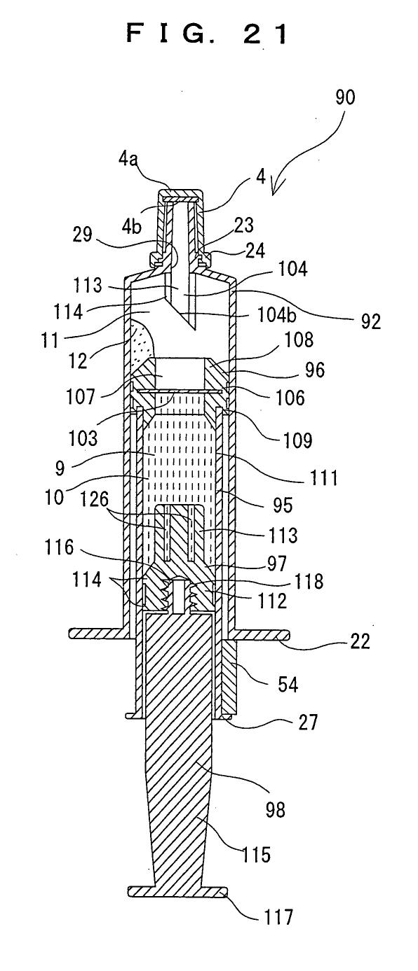

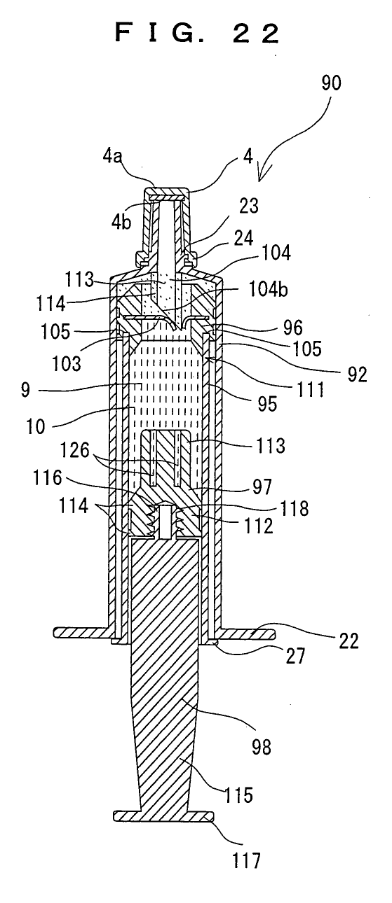

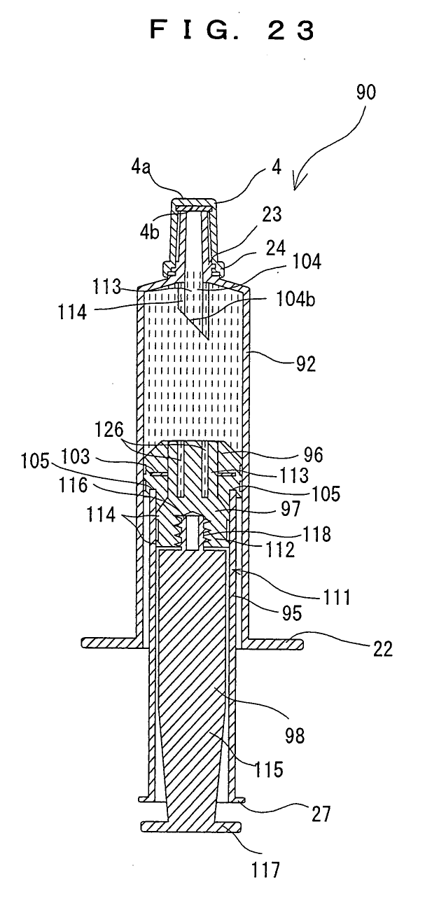

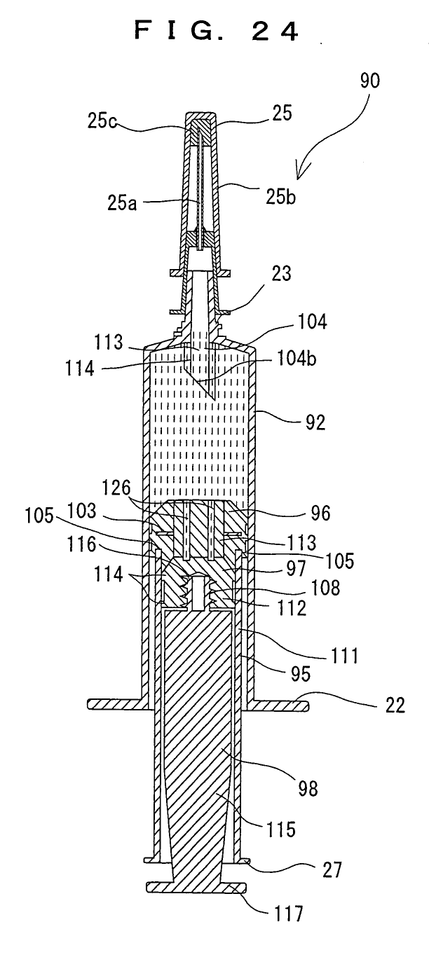

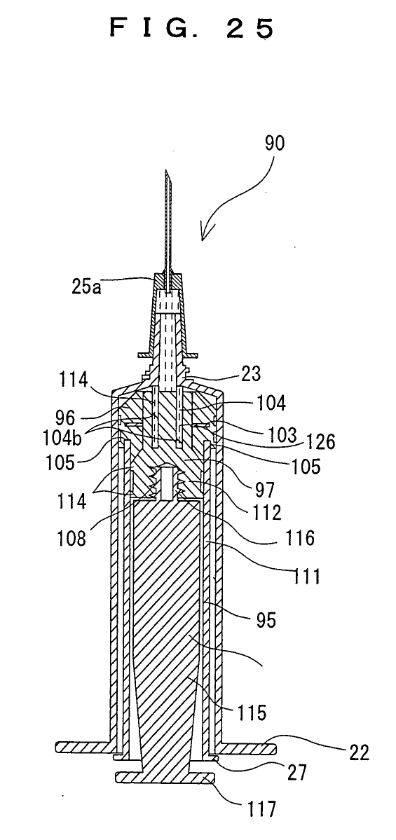

FIG. 20 is an outlook view of the two-chamber type pre-filled syringe according to another embodiment of the present invention. FIG. 21 is a sectional view of the two-chamber type pre-filled syringe shown in FIG. 20. FIG. 22 is an explanatory view for explaining the operation of the two-chamber type pre-filled syringe shown in FIG. 20. FIG. 23 is an explanatory view for explaining the operation of the two-chamber type pre-filled syringe shown in FIG. 20. FIG. 24 is an explanatory view for explaining the operation of the two-chamber type pre-filled syringe shown in FIG. 20. FIG. 25 is an explanatory view for explaining the operation of the two-chamber type pre-filled syringe shown in FIG. 20.

A two-chamber type pre-filled syringe 90 of the present invention includes an outer cylinder 92 having a needle-mounting portion 21 and a projected portion 104 for breaking use disposed inside the outer cylinder 92 and projecting toward a rear end thereof; closing members 4, 25 mounted on the needle-mounting portion 21; an inner cylinder assemblage 111 having a cylindrical inner cylinder 95 accommodated inside the outer cylinder 92 and being open at both ends thereof, a cylindrical first gasket 96 provided in the vicinity of a front-end portion of the inner cylinder 95 and slidable inside the outer cylinder 92, and a sealing member 103 which airtightly seals a through-hole (in other words, path) 107 of the first gasket 96 and can be broken by the projected portion 104 for breaking use provided inside the outer cylinder 92; a second gasket 97 slidably accommodated inside the inner cylinder 95; a plunger 98 which is mounted or can be mounted at a rear-end portion of the second gasket 97; a first accommodation portion 9 formed among the sealing member 103, the second gasket 97, and the inner cylinder 95; a second accommodation portion 11 formed between the inner cylinder assemblage 111 and the outer cylinder 92; a medicine-dissolving liquid 10 accommodated in the first accommodation portion 9; and a powdery or frozen dry medicine 12 accommodated inside the second accommodation portion 11.

The main difference between the two-chamber type pre-filled syringe 90 of this embodiment and the above-described two-chamber type pre-filled syringe 1 is that in the pre-filled syringe 1, the front end opening of the inner cylinder 5 is airtightly sealed, whereas in the pre-filled syringe 90, the inside of the path 107 of the first gasket 96 is airtightly sealed and the configuration of the outer cylinder 92 is different from that of the outer cylinder 2.

The outer cylinder 2 is cylindrical. The needle-mounting portion 21 being open at its front end and tapered toward its front end is disposed at the front end of the outer cylinder 2. A pair of flanges 22 is disposed at the rear end of the outer cylinder 2, with the flanges 22 confronting each other. A male screw portion 23 that engages a female screw portion 24 formed on the inner peripheral surface of the sealing cap 4 is formed on the outer surface of the base portion of the needle-mounting portion 21. A constituent material similar to that of the outer cylinder 2 is used for the constituent material of the outer cylinder 92. To keep a dry state, it is preferable to make the outer cylinder comparatively thick. It is preferable that the length of the outer cylinder 92 is 50 to 200 mm. It is preferable that the volume of the outer cylinder 92 is 1.0 to 60 ml. The sealing cap 4 is mounted on the needle-mounting portion 21 of the outer cylinder 92. As the sealing cap 4, the same one as the above-described one is used.

The projected member 104 for breaking use projects from the vicinity of the inner open portion 29 of the needle-mounting portion 21 of the outer cylinder 92 to the rear-end side of the outer cylinder 92 and has a sealing member-breaking blade portion 104b at the rear end thereof. More specifically, the projected portion 104 for breaking use is hollow and has a medicine guide path 113 therein. The projected portion 104 for breaking use extends from the vicinity of the rear end of the needle-mounting portion 21 toward the rear end of the outer cylinder and is coaxial with the needle-mounting portion 21.

A slit 114 extending from a front end opening (rear side) of the projected portion 104 for breaking use to the base thereof or the vicinity of the base is formed on a side surface of the projected portion 104 for breaking use. The slit 114 communicates with the medicine guide path 113. Two slits 114 are formed in confrontation with the side surface of the projected portion 104 for breaking use. Owing to this construction, the liquid medicine inside the syringe 90 is capable of flowing from the front end opening of the projected portion 104 for breaking use and the slit 114 to the needle-mounting portion 21.

By constructing the two-chamber type pre-filled syringe as described above, the projected portion 104 for breaking use penetrates through the sealing member 103 owing to the movement of the inner cylinder assemblage 111 toward the front-end side of the outer cylinder 92 or the movement of the outer cylinder 92 toward the rear-end side of the inner cylinder assemblage 111, thus allowing communication between the first accommodation portion 9 and the second accommodation portion 11.

As shown in FIGS. 20 and 21, the inner cylinder assemblage 111 includes the inner cylinder 95, the first gasket 96, and the sealing member 103.

As shown in FIG. 20, the inner cylinder 95 is open at its front and rear ends and has entirely almost the same diameter. The front end opening of the inner cylinder 95 is airtightly mounted at the rear-end portion of the first gasket 96. The flange 27 is formed at the rear end of the inner cylinder 95. As the constituent material for the inner cylinder 95, it is preferable to use a constituent material similar to that of the inner cylinder 5. To keep a dry state of the medicine 12, it is preferable to make the inner cylinder comparatively thick. It is preferable that the length of the inner cylinder 95 is 5 to 200 mm. It is preferable that the volume of the inner cylinder 95 is 1 to 60 ml.

As shown in FIGS. 20 and 21, the first gasket 96 is cylindrical, mounted on the front end opening of the inner cylinder 95, and has a through-hole (in other words, path) 107 allowing communication between the inside of the inner cylinder 95 and the inside of the outer cylinder 92 and allowing the projected portion 104 for breaking use to enter thereinto. The constituent material for the first gasket 96 is similar to that of the first gasket 6.

The first gasket 96 has a body portion 106 extending in almost the same diameter, a front-end portion 108 disposed at the front end of the body portion 106 in such a way that the front-end portion 108 taperingly decreases in its diameter toward the front-end side of the outer cylinder 92, three annular ribs 109 liquid-tightly contacting the outer cylinder 92 disposed on the peripheral surface of the body portion, and the sealing member 103 that airtightly seals the vicinity of the intermediate portion of the through-hole 107 inside the first gasket 96. At the rear-end portion of the first gasket 96, an annular concavity 105 is provided. A front end opening portion of the inner cylinder 95 is inserted into the concavity 105 and airtightly fixed thereto by an adhesive agent, thermal fusion, high-frequency fusion or the like. As shown in FIG. 23, the configuration of the rear-end surface of the front-end portion of the first gasket 96 corresponds to that of the front-end surface of the second gasket 97 to prevent a gap from being generated therebetween as much as possible.

The sealing member 103 is formed circularly and thinly. The periphery of the sealing member 103 is fixed to the inside of the first gasket 96, thus airtightly sealing the through-hole 107 of the first gasket 96. The sealing member 103 is formed in a manner similar to that of forming the sealing member 13. The sealing member 103 can be fixed to the first gasket 96 by disposing the sealing member 103 in a die for forming the first gasket 96 at a position thereof across the vicinity of the center of the through-hole 107 and then performing insert molding by injecting a constituent material for the first gasket or fixing the sealing member 103 to a path of the first gasket 96 by means of thermal fusing, high-frequency fusing or ultrasonic wave fusing. The sealing member may be so constructed as to airtightly seal the front end opening of the first gasket or the rear end opening thereof. In this case, it is preferable to fix the sealing member to the front end opening of the inner cylinder or the rear end opening thereof by means of thermal fusing, high-frequency fusing or ultrasonic wave fusing. The sealing member is thin in the embodiment of the present invention. But provided that the projected portion for breaking use is capable of penetrating through the sealing member, the sealing member may be thick to some extent.

The second gasket 97 has a body portion 112 extending in an equal outer diameter, a projected portion 113 which is provided at the front-end portion of the body portion 112 and capable of penetrating into the through-hole 107 of the first gasket 96 from the rear-end side thereof, two annular ribs 114 liquid-lightly contacting the inner cylinder 95 provided on the peripheral surface of the body portion 112. At the rear-end portion of the second gasket 97, a female screw portion 118 engaging a male screw portion formed at the front-end portion of the plunger 98 is provided.

The projected portion 113 is formed in the shape of a column having a small diameter than that of the body portion 112. The outer diameter of the projected portion 113 is set almost equally to or a little smaller than the inner diameter of the through-hole 107 of the first gasket 96. As shown in FIG. 23, the length of the projected portion 113 is so set that the front end of the projected portion 113 is disposed in the vicinity of the front-end of the first gasket 96, when the front-end portion of the second gasket 97 is accommodated in the first gasket 96. A material similar to that of the above-described second gasket 7 is used as the constituent material of the second gasket 97.

The second gasket 97 has an accommodation portion 126 which is formed in the direction from the front end thereof to the rear-end side and capable of accommodating the projected portion 104 for breaking use partly or entirely. As shown in FIGS. 21 and 25, the accommodation portion 126 is formed as a conic concavity capable of accommodating almost the entire projected portion 104 for breaking use without a gap. By forming the accommodation portion 126 in the second gasket 97, it is possible to reduce formation of a dead space when the plunger 98 is pressed completely to the front-end side and hence reduce a residual amount of the liquid medicine. A material similar to that of the first gasket 96 is used as the constituent material of the second gasket 97.

The plunger 98 has a body portion 115 cross-shaped in section and extending axially, a male screw portion 116 formed at the front end of the body portion 115 and engaging a female screw portion 118 formed at the rear-end portion of the second gasket 97, and disk-shaped portion 117 for pressing use disposed at the rear end of the body portion 115.

The two-chamber type pre-filled syringe 90 has the first accommodation portion 9 formed among the first gasket 96, the rear portion of the sealing member 103, the front portion of the second gasket 97, and the inner peripheral surface of the inner cylinder; the medicine-dissolving liquid 10 accommodated in the first accommodation portion 9; the second accommodation portion 11 formed among the front portion of the first gasket 96, the front portion of the sealing member 93 (inner cylinder assemblage 111), and the inner peripheral surface of the outer cylinder; and the powdery or frozen dry medicine 12 accommodated inside the second accommodation portion 11. The volume of the first accommodation portion and that of the second accommodation portion are as described above. The powdery or frozen dry medicine 12 and the medicine-dissolving liquid 10 are as described above. The first accommodation portion 9 may be formed as a decompressed space. By doing so, a liquid injection work can be facilitated. In this case, it is necessary to provide the inner cylinder stopping mechanism 54 for preventing the inner cylinder 95 from moving toward the front end of the outer cylinder 92. The inner cylinder stopping mechanism 54 is a plate-shaped member contacting the rear-end surface of the flange 22 of the outer cylinder 92 and the front-end surface of the flange 27 of the inner cylinder 95. Unless the inner cylinder stopping mechanism 54 is removed, the inner cylinder 95 does not move to the front end side. The pre-filled syringe of the present invention may have a function of preventing removal of the plunger by joining the flange of the inner cylinder or that of the outer cylinder and the plunger with each other with a mountable stopper.

The operation of the above-described two-chamber type pre-filled syringe 90 is described below with reference to FIGS. 21 through 25.

An inner cylinder stopping mechanism 54 is removed from the two-chamber type pre-filled syringe 90 placed in a state shown in FIG. 21. When the plunger 98 is gradually pressed toward the front-end side of the outer cylinder, the inner cylinder assemblage 111 moves toward the front-end side of the outer cylinder, and the rear end of the projected portion 104 for breaking use breaks through the sealing member 103 of the first gasket 96, as shown in FIG. 22. As a result, the first accommodation portion 9 and the second accommodation portion 11 communicate with each other. By further pressing the plunger 98 toward the front-end side of the outer cylinder, the medicine-dissolving liquid 10 accommodated in the first accommodation portion 9 shifts into the second accommodation portion 11, and the first gasket 96 moves to the rear-end side of the outer cylinder. Then the syringe 90 is shaken to securely dissolve the medicine in the medicine-dissolving liquid 10.

Thereafter the sealing cap 4 is removed from the needle-mounting portion 21, and the needle 25 is mounted on the needle-mounting portion 21 of the outer cylinder 92. Thereby as shown in FIG. 23, preparation for administering the liquid medicine is completed. Then the cap 25b of the needle 25 is removed. After air inside the syringe 90 is eliminated, the syringe 90 is pierced into a necessary portion of a patient. After air inside the syringe 90 is eliminated, the syringe 90 is pierced into a necessary portion of a patient. By pressing the plunger 98, the liquid medicine inside the syringe 90 is administered. In a state where the plunger 98 is pressed completely to the front-end side of the outer cylinder, as shown in FIG. 23, the entirety of the hollow needle-shaped projected portion 104 for breaking use is accommodated in the accommodation portion 126 of the second gasket 97. Therefore it is difficult for the liquid medicine to remain inside the syringe 90. When the sealing cap is of a type in which the double ended needle is mounted on the needle-mounting portion, a medicine may be administered with the double ended needle mounted on the sealing cap without removing the sealing cap from the needle-mounting portion.

### INDUSTRIAL APPLICABILITY

The two-chamber type pre-filled syringe of the present invention has the outer cylinder having the needle-mounting portion and the projected portion for breaking use provided inside the outer cylinder and projecting toward the rear end thereof; the closing member mounted on the needle-mounting portion; the cylinder assemblage comprising the cylindrical inner cylinder accommodated inside the outer cylinder and being open at both ends thereof, the annular first gasket provided in the vicinity of the front-end portion of the inner cylinder and slidable inside the outer cylinder, and the sealing member which airtightly seals the front-end portion of the inner cylinder or the path of the first gasket and can be broken by the projected portion for breaking use provided inside the outer cylinder; the second gasket sidably accommodated inside the inner cylinder; the plunger which is mounted or can be mounted at the rear-end portion of the second gasket; the first accommodation portion formed among the sealing member, the second gasket, and the inner cylinder; the second accommodation portion formed between the inner cylinder assemblage and the outer cylinder; the medicine-dissolving liquid accommodated in the first accommodation portion; and the powdery or frozen dry medicine accommodated inside the second accommodation portion.

Therefore the two-chamber pre-filled syringe makes it difficult for a liquid or the like to leak therefrom during transport and preparation for use. Further the two-chamber pre-filled syringe allows a mixing operation to be performed easily and securely when it is used.

1. A two-chamber type pre-filled syringe comprising:
an outer cylinder having a needle-mounting portion at a front-end side thereof and a projected portion for breaking use provided inside said outer cylinder at a front-end side thereof and projecting toward a rear end thereof;
a closing member mounted on said needle-mounting portion;
an inner cylinder assemblage comprising a cylindrical inner cylinder accommodated inside said outer cylinder and being open at both ends thereof, an annular first gasket provided in the vicinity of a front-end portion of said inner cylinder and slidable inside said outer cylinder, and a sealing member which airtightly seals a front-end portion of said inner cylinder or a path of said first gasket and can be broken by said projected portion for breaking use provided inside said outer cylinder;
a second gasket slidably accommodated inside said inner cylinder;
a plunger which is mounted or can be mounted at a rear-end portion of said second gasket;
a first accommodation portion formed among said sealing member, said second gasket, and said inner cylinder;
a second accommodation portion formed between said inner cylinder assemblage and said outer cylinder;
a medicine-dissolving liquid accommodated in said first accommodation portion; and
a powdery or frozen dry medicine accommodated inside said second accommodation portion.

2. A two-chamber type pre-filled syringe according to claim 1, wherein said projected portion for breaking use penetrates through said sealing member owing to a movement of said inner cylinder assemblage toward said front-end side of said outer cylinder or a movement of said outer cylinder toward a rear-end side of said inner cylinder assemblage, thereby allowing communication between said first accommodation portion and said second accommodation portion.

3. A two-chamber type pre-filled syringe according to claim 1, wherein said first gasket is annular, provided in the vicinity of said front-end portion of said inner cylinder, and has a through-hole communicating an inside of said inner cylinder and an inside of said outer cylinder with each other and allowing said projected portion for breaking use to enter thereinto.

4. A two-chamber type pre-filled syringe according to claim 1 wherein said sealing member is a film having a property of being vapor-unpermeable therethrough or a property of being hard for vapor to permeate therethrough.

5. A two-chamber type pre-filled syringe according to claim 1, wherein said projected portion for breaking use projects from a neighborhood of an inner open portion of a needle-mounting portion of said outer cylinder toward a rear-end side of said outer cylinder and has a sealing member-breaking pointed-end portion or a blade portion disposed at a rear end of said projected portion for breaking use.

6. A two-chamber type pre-filled syringe according to claim 1, wherein said projected portion for breaking use has a liquid medicine guide path for guiding a liquid medicine from a side of said projected portion for breaking use to a side of said needle-mounting portion.

7. A two-chamber type pre-filled syringe according to claim 1, wherein said two-chamber type pre-filled syringe has a projected member for breaking use mounted on an inner open portion of said needle-mounting portion disposed inside said outer cylinder, wherein said projected member for breaking use has a projected portion for breaking use extending toward a rear-end side of said outer cylinder, a mounting portion to be mounted on said outer cylinder, and a liquid medicine guide path for guiding a liquid medicine from a side of said projected portion for breaking use to a side of said needle-mounting portion.

8. A two-chamber type pre-filled syringe according to claim 1, wherein said sealing member is formed to seal a front end opening of said inner cylinder.

9. A two-chamber type pre-filled syringe according to claim 1, wherein said second gasket has an accommodation portion which is formed in a direction from a front end thereof to a rear-end side and capable of accommodating said projected portion for breaking use partly or entirely.
